(19) United States
(12) Patent Application Publication
(10) Pub. No.: US 2021/0032666 A1
(43) Pub. Date: Feb. 4, 2021

(54) METHOD FOR PRODUCING COENZYME Q10

(71) Applicant: KANEKA CORPORATION, Osaka-shi, Osaka (JP)

(72) Inventor: Yasuyuki SUZUKI, Takasago-shi (JP)

(73) Assignee: KANEKA CORPORATION, Osaka-shi, Osaka (JP)

(21) Appl. No.: 17/042,570

(22) PCT Filed: Mar. 27, 2019
(86) PCT No.: PCT/JP2019/013065

§ 371 Date: Sep. 28, 2020

(30) Foreign Application Priority Data
Mar. 28, 2018 (JP) 2018-062841

(51) Int. Cl. C12P 7/66

(52) U.S. Cl. CPC C12P 7/66

(57) ABSTRACT

Methods for stably producing coenzyme Q10 are provided, which efficiently remove impurities derived from a microorganism in an extract of a coenzyme Q10-producing microorganism. A method for producing coenzyme Q10 includes: cooling a hydrophobic organic solvent extract of a coenzyme Q10-producing microorganism or a concentrated liquid of the hydrophobic organic solvent extract; and separating/removing the solid precipitated in the cooling step, wherein the hydrophobic organic solvent extract or the concentrated liquid has a water content of 50 ppm by weight or more and 1% by weight or less. Preferably, the extract obtained by extraction from a coenzyme Q10-producing microorganism in an organic solvent is brought into contact and mixed with an aqueous alkaline solution and then washed with water, where a resulting extract is concentrated to obtain the concentrated liquid, and the concentrated liquid is subjected to the cooling step.

### TECHNICAL FIELD

The present invention relates to a method for producing coenzyme Q10. More specifically, the present invention relates to a method for producing coenzyme Q10 including cooling a hydrophobic organic solvent extract of a microorganism and separating and removing a precipitated solid.

### BACKGROUND ART

Coenzyme Q is an essential component which is widely distributed in living organisms, from bacterium to mammals and is known as a constituent of an electron transport system of mitochondria in living body cells. Coenzyme Q repeats an oxidation and reduction to play a role as a messenger component in an electron transport system in a mitochondria. In addition, it is known that reduced coenzyme Q has an antioxidant property. Human coenzyme Q contains coenzyme Q10 having 10 repetitive structures in the side chain as a main component, and generally about 40 to 90% of human coenzyme Q exists in a reduced state in a living body. A physiological function of coenzyme Q is exemplified by an activation of an energy production by activating a mitochondria, an activation of cardiac function, a stabilization of a cell membrane, and a protection of cells by antioxidant action.

Many of the coenzyme Q10 which is currently manufactured and marketed are oxidized type, but recently reduced coenzyme Q10 having higher oral absorbability than oxidized coenzyme Q10 has been marketed and has been widely used.

Several methods for producing coenzyme Q10 have been known. For example, Patent document 1 discloses a method for producing reduced coenzyme Q10 including a step of holding a solution containing reduced coenzyme Q10 at temperatures exceeding 47° C. for at least 60 minutes, and a step of crystallization (specifically, cooling crystallization, poor solvent crystallization, or a combination of cooling crystallization and other crystallization methods) thereafter.

In addition, Patent document 2 discloses a method for producing coenzyme Q10 by repeating an extraction treatment in which coenzyme Q10-containing substance is contacted with a hydrophilic solvent in the presence of water and an adsorption treatment to adsorb coenzyme Q10 in the coenzyme Q10 extract obtained by the extraction treatment on a hydrophobic adsorbent.

Patent document 3 discloses a method for producing coenzyme Q10 including the step of brining an extract of a coenzyme Q10-producing microorganism into contact with an adsorbent containing aluminum silicate as a main component singly or with the adsorbent and a different adsorbent in combination. As the adsorbent containing aluminum silicate as a main component, for example, activated clay or the like is used. Patent document 3 describes that, according to the method of Patent document 3, it is possible to provide a method for stably producing coenzyme Q10 with a simple coenzyme Q10 production step by efficiently removing impurities derived from a microorganism from an extract of a coenzyme Q10-producing microorganism.

Furthermore, Patent document 4 discloses a method for purifying coenzyme Q10 in which coenzyme Q10 is extracted from photosynthetic bacteria cells containing coenzyme Q10 in a hydrophilic organic solvent, the water content of the extract is adjusted, and the extract is cooled to precipitate and collect coenzyme Q10.

### PRIOR ART DOCUMENT

### Patent Document

Patent document 1: JP 2015-131766 A
Patent document 2: JP S59-173088 A
Patent document 3: WO 2018/003974 A1
Patent document 4: JP S57-63094 A

### SUMMARY OF THE INVENTION

### Problems to be Solved by the Invention

However, there is still a need for easy and stable mass production of coenzyme Q10 at a low cost in the above-described conventional methods.

For example, with respect to the method of Patent document 1, when a large amount of impurities coexists in an extract of a coenzyme Q10-producing microorganism, it is difficult to obtain coenzyme Q10 having a high purity by crystallization only. Even when coenzyme Q10 can be obtained by purification by crystallization, the operating conditions such as crystallization operating temperature must be strictly controlled, and the time required for the crystallization process may be prolonged.

With respect to the adsorption method of Patent document 2, since the purpose of the method is to adsorb coenzyme Q10 itself on an adsorbent, the step of separating and eluting coenzyme Q10 from the adsorbent by using an elution solvent is required after the adsorption treatment to obtain coenzyme Q10.

With respect to the method of Patent document 3, an adsorbent is used in order to remove impurities. However, the method has many problems to be solved, such as an increase in raw material cost accordingly, an increase in the amount of waste associated with the disposal of the adsorbent used for the adsorption treatment, and problems of equipment and energy for regenerating the adsorbent.

With respect to the method of Patent document 4, a coenzyme Q10-producing microorganism is limited to photosynthetic bacteria having a small amount of fat-soluble impurities. Even in this method, there is a problem, for example, that the yield must be sacrificed in order to obtain highly-pure coenzyme Q10.

The objective of the present invention to solve the above-described problems is to provide a method for stably producing coenzyme Q10 in a high yield with a simple coenzyme Q10 production step by efficiently removing impurities derived from a microorganism in an extract of a coenzyme Q10-producing microorganism.

### Solution to the Problems

The inventor of the present invention intensively studied ways for solving the above problems. As a result, the present inventor found that, by cooling a hydrophobic extract of a coenzyme Q10-producing microorganism or a concentrated liquid of the hydrophobic organic solvent extract (hereinafter sometimes referred to as an extract) having a water content of 50 ppm by weight or more and 1% by weight or less, impurities other than coenzyme Q10 are precipitated as a solid, and a separation step of separating and removing the precipitated solid is conducted, whereby coenzyme Q10 giving a high yield can be efficiently purified without using auxiliary materials such as activated clay. The present invention has been made based on such a finding.

The configuration of the method for producing coenzyme Q10 according to the present invention is as follows.

1. A method for producing coenzyme Q10 comprising:
a cooling step of cooling a hydrophobic organic solvent extract of a coenzyme Q10-producing microorganism or a concentrated liquid of the hydrophobic organic solvent extract; and
a separation step of separating and removing a solid precipitated in the cooling step, wherein
the hydrophobic organic solvent extract or the concentrated liquid has a water content of 50 ppm by weight or more and 1% by weight or less.
2. The method according to the above 1, wherein the hydrophobic organic solvent extract of the coenzyme Q10-producing microorganism is brought into contact and mixed with an aqueous alkaline solution and then washed with water, a resulting extract is concentrated to obtain the concentrated liquid, and the concentrated liquid is subjected to the cooling step.
3. The method according to the above 2 comprising:
repeating a procedure in which, after the solid obtained in the separation step is added to the extract before being brought into contact and mixed with the aqueous alkaline solution, a resulting extract is brought into contact and mixed with an aqueous alkaline solution, washed with water, and then subjected to the cooling step, or
repeating a procedure in which, after the solid obtained in the separation step is added to the extract after being brought into contact and mixed with the aqueous alkaline solution, a resulting extract is washed with water and subjected to the cooling step.
4. The method according to any one of the above 1 to 3, wherein a temperature of the cooling in the cooling step is 20° C. or lower.
5. The method according to any one of the above 1 to 4, wherein a concentration of coenzyme Q10 in the extract or the concentrated liquid at the time of the cooling in the cooling step is 0.1 g/L or more and 300 g/L or less.
6. The method according to any one of the above 1 to 5, wherein the hydrophobic organic solvent is a hydrocarbon solvent and/or a fatty acid ester solvent.
7. The method according to any one of the above 1 to 6, wherein the solid in the separation step is separated and removed from the extract or the concentrated liquid by a rotary filter.

### Advantageous Effects of the Invention

According to the present invention, by cooling a hydrophobic organic solvent extract of a microorganism containing coenzyme Q10 and separating a precipitated solid, impurities can be easily removed, and thus high-quality coenzyme Q10 can be obtained in a high yield and successfully in terms of workability and economy.

### DESCRIPTION OF EMBODIMENTS

Hereinafter, one embodiment of the method for producing coenzyme Q10 according to the present invention is described, but the present invention is not restricted thereto.

The production method of the present invention is characterized by including a cooling step of cooling a hydrophobic organic solvent extract of a coenzyme Q10-producing microorganism or a concentrated liquid of the hydrophobic organic solvent extract; and a separation step of separating and removing a solid precipitated in the cooling step, wherein the hydrophobic organic solvent extract or the concentrated liquid has a water content of 50 ppm by weight or more and 1% by weight or less.

### (1) Coenzyme Q10-Producing Microorganism Used in the Present Invention

Coenzyme Q10 includes an oxidized type and a reduced type. The target of the present invention is both of oxidized coenzyme Q10 and reduced coenzyme Q10 as coenzyme Q10, and coenzyme Q10 containing both of oxidized coenzyme Q10 and reduced coenzyme Q10 is also the target of the present invention. When coenzyme Q10 is a mixture of oxidized coenzyme Q10 and reduced coenzyme Q10, a content ratio of reduced coenzyme Q10 is not particularly restricted. The description of mere “coenzyme Q10” in this disclosure represents any of oxidized coenzyme Q10, reduced coenzyme Q10, and a mixture of oxidized coenzyme Q10 and reduced coenzyme Q10.

As the coenzyme Q10-producing microorganism usable in the present invention, any one of bacteria, yeast and fungus may be used without limitation as long as the microorganism can produce coenzyme Q10 in the microorganism. Such a microorganism is specifically exemplified by a microorganism belonging to genera of Acetobacter, Aminobacter, Agromonas, Acidiphilium, Bulleromyces, Bullera, Brevundimonas, Cryptococcus, Chionosphaera, Candida, Cerinosterus, Exisophiala, Exobasidium, Fellomyces, Filobasidiella, Filobasidium, Geotrichum, Graphiola, Gluconobacter, Kockovaella, Kurtzmanomyces, Lalaria, Leucosporidium, Legionella, Methylobacterium, Mycoplana, Oosporidium, Pseudomonas, Psedozyma, Paracoccus, Petromyc, Rhodotorula, Rhodosporidium, Rhizomonas, Rhodobium, Rhodoplanes, Rhodopseudomonas, Rhodobacter, Sporobolomyces, Sporidiobolus, Saitoella, Schizosaccharomyces, Sphingomonas, Sporotrichum, Sympodiomycopsis, Sterigmatosporidium, Tapharina, Tremella, Trichosporon, Tilletiaria, Tilletia, Tolyposporium, Tilletiopsis, Ustilago, Udeniomyce, Xanthophllomyces, Xanthobacter, Paecilomyces, Acremonium, Hyhomonus, Rhizobium, Phaffia and Haematococcus.

Among them, from the aspect of easy cultivation and productivity, bacteria and yeast are preferred. As bacteria, non-photosynthetic bacteria is preferred, and further, bacteria belonging to genera of Agrobacterium and Gluconobacter are particularly preferred. In addition, as yeast, a yeast belonging to genera of Schizosaccharomyces, Saitoella and Phaffia are particularly preferred.

When reduced coenzyme Q10 is purposely produced as coenzyme Q10, it is preferred to use a microorganism by which produced coenzyme Q10 has high content ratio of reduced coenzyme Q10. For example, it is more preferred to use a microorganism of which content ratio by weight of reduced coenzyme Q10 in coenzyme Q10 after cultivation is preferably 70% or more and more preferably 80% or more.

As the coenzyme Q10-producing microorganism used in the present invention, not only a wild strain of the above-described microorganism but also a variant and a recombinant of the above-described microorganism of which transcription activity and translation activity of a gene involved in a biosynthesis of the target coenzyme Q10 and an enzyme activity of an expressed protein is altered or improved can be used.

By cultivating the above-described microorganism, microorganism cells containing coenzyme Q10 can be obtained. A cultivating method is not particularly restricted, and a cultivating method suitable for the target microorganism or the production of the target coenzyme Q10 can be appropriately selected. A cultivation time is also particularly not restricted, and may be adjusted to the range that a desired amount of the target coenzyme Q10 is accumulated in microorganism cells.

As the method for extracting coenzyme Q10 from the above-described microorganism cells in the production method of the present invention, coenzyme Q10 can be directly extracted from the microorganism cells, or the microorganism cells are homogenized to obtain a microorganism cell homogenate or an aqueous dispersion of a microorganism cell homogenate as a pretreatment, and coenzyme Q10 can be extracted from the obtained homogenate or the aqueous dispersion of the microorganism cell homogenate. Alternatively, the microorganism cells are dried as a pretreatment, and coenzyme Q10 can be extracted from the dried microorganism cells. In the “homogenization” in the present invention, the surface structure of a cell wall or the like is damaged so that it becomes possible to extract the target coenzyme Q10.

The homogenization method used in the present invention is exemplified by physical treatment and chemical treatment.

The above-described physical treatment is exemplified by a treatment using high pressure homogenizer, rotary blade homogenizer, ultrasonic homogenizer, French press, ball mill or the like, and a combination thereof.

The above-described chemical treatment is exemplified by a treatment using an acid such as hydrochloric acid and sulfuric acid, preferably a strong acid, a base such as sodium hydroxide and potassium hydroxide, preferably a strong base, or a combination thereof.

As a method for homogenizing cells as a pretreatment for the extraction and recovery of coenzyme Q10 in the present invention, a physical treatment is more preferred among the above-described homogenization methods in terms of a homogenization efficiency

A form of the microorganism cells used for the above-described cell homogenization may be a culture medium, a concentrated culture medium, wet microorganism, washed wet microorganism, or a wet microorganism dispersion (for example, including water, a saline solution and a buffer solution), preferably an aqueous dispersion of the microorganism cells, and more preferably a culture medium, a concentrated culture medium, a washed culture medium, and a washed concentrated culture medium in terms of handling property.

A microorganism concentration in the aqueous dispersion of the microorganism cell homogenate is not particularly restricted, and the concentration in terms of the weight of the dried microorganism is generally in a range of 1 to 25 wt %, and preferably in a range of 10 to 20 wt % from a viewpoint of economy.

### (2) Extraction of Coenzyme Q10 from Coenzyme Q10-Producing Microorganism

Coenzyme Q10 is extracted from the above-described coenzyme Q10-producing microorganism using an organic solvent. Specifically, in the present invention, although it is necessary to use a hydrophobic organic solvent as a solvent (solvent of an extract or a concentrated liquid at the time of cooling) used at the time of cooling in a cooling step described later, an organic solvent used for extraction of coenzyme Q10 (extraction of a microorganism-derived component) from a coenzyme Q10-producing microorganism is not particularly restricted, and a hydrophobic organic solvent or a hydrophilic organic solvent can be used. It is preferred to use a hydrophobic organic solvent at the time of the extraction because the extract and the concentrate thereof can be directly subjected to the cooling step.

Examples of the organic solvent used for the extraction of coenzyme Q10 in the present invention include a hydrocarbon solvent, a fatty acid ester solvent, an ether solvent, an alcohol solvent, a fatty acid solvent, a ketone solvent, a nitrogen compound solvent such as a nitrile solvent and an amide solvent, and a sulfur compound solvent.

A hydrocarbon solvent is not particularly restricted and is exemplified by an aliphatic hydrocarbon solvent, an aromatic hydrocarbon solvent and a halogenated hydrocarbon solvent. Among the examples, an aliphatic hydrocarbon solvent and an aromatic hydrocarbon solvent are preferred, and an aliphatic hydrocarbon solvent is more preferred.

An aliphatic hydrocarbon solvent may be cyclic or non-cyclic and saturated or unsaturated, is not particularly restricted, and a saturated aliphatic hydrocarbon solvent is generally used. In general, a C₃₋₂₀ aliphatic hydrocarbon solvent is used, a C₅₋₁₂ aliphatic hydrocarbon solvent is preferably used, and a C₅₋₈ aliphatic hydrocarbon solvent is more preferably used. Specifically, propane, butane, isobutane, pentane, 2-methylbutane, hexane, 2-methylpentane, 2,2-dimethylbutane, 2,3-dimethylbutane, heptane, a heptane isomer such as 2-methylhexane, 3-methylhexane, 2, 3-dimethylpentane and 2, 4-dimethylpentane, octane, 2,2,3-trimethylpentane, isooctane, nonane, 2,2,5-trimethylhexane, decane, dodecane, 2-pentene, 1-hexene, 1-heptene, 1-octene, 1-nonene, 1-decene, cyclopentane, methylcyclopentane, cyclohexane, methylcyclohexane, ethylcyclohexane, p-menthane and cyclohexene are exemplified. Preferably, pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, nonane, 2,2,5-trimethylhexane, decane, dodecane, cyclopentane, methylcyclopentane, cyclohexane, methylcyclohexane, ethylcyclohexane and p-menthane are exemplified. More preferably, pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane and ethylcyclohexane are exemplified. More preferably, pentane, hexane, cyclohexane and methylcyclohexane are exemplified. Particularly preferably, heptane, hexane and methylcyclohexane are exemplified, in view of very high protection effect from oxidation and versatility. Most preferably, heptane and hexane are exemplified.

An aromatic hydrocarbon solvent is not particularly restricted, and a C₆₋₂₀ aromatic hydrocarbon solvent is generally used, a C₆₋₁₂ aromatic hydrocarbon solvent is preferably used, and a C₇₋₁₀ aromatic hydrocarbon solvent is more preferably used. Specifically, an aromatic hydrocarbon solvent is exemplified by benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene and styrene, preferably toluene, xylene, o-xylene, m-xylene, p-xylene, ethylbenzene, cumene, mesitylene, tetralin, butylbenzene, p-cymene, cyclohexylbenzene, diethylbenzene and pentylbenzene, more preferably toluene, xylene, o-xylene, m-xylene, p-xylene, cumene and tetralin, and most preferably cumene.

A halogenated hydrocarbon solvent may be cyclic or non-cyclic and saturated or unsaturated, is not particularly restricted, and a non-cyclic halogenated hydrocarbon solvent is preferably used. A halogenated hydrocarbon solvent is more preferably a chlorinated hydrocarbon and a fluorinated hydrocarbon, and even more preferably a chlorinated hydrocarbon. In addition, a C₁₋₆ halogenated hydrocarbon solvent may be used, a C₁₋₄ halogenated hydrocarbon solvent is preferably used, and a C₁₋₂ halogenated hydrocarbon solvent is more preferably used. Specifically, a halogenated hydrocarbon solvent is exemplified by dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2 -tetrachloroethane, pentachloroethane, hexachloroethane, 1,1-dichloroethylene, 1,2-dichloroethylene, trichloroethylene, tetrachloroethylene, 1,2 -dichloropropane, 1,2,3-trichloropropane, chlorobenzene and 1,1,1,2-tetrafluoroethane, preferably dichloromethane, chloroform, carbon tetrachloride, 1,1-dichloroethane, 1,2-dichloroethane, 1,1,1-trichloroethane, 1,1,2-trichloroethane, 1,1-dichloroethylene, 1,2-dichloroethylene, trichloroethylene, chlorobenzene and 1,1,1,2-tetrafluoroethane, more preferably dichloromethane, chloroform, 1,2-dichloroethylene, trichloroethylene, chlorobenzene and 1,1,1,2-tetrafluoroethane.

A fatty acid ester solvent is not particularly restricted, and is exemplified by a propionate ester, an acetate ester and a formate ester, preferably an acetate ester and a formate ester, and more preferably an acetate ester. An ester group is not particularly restricted, and a C₁₋₈ alkyl ester and a C₇₋₁₂ aralkyl ester are generally used, a C₁₋₆ alkyl ester is preferably used, and a C₁₋₄ alkyl ester is more preferably used.

A propionate ester is specifically exemplified by methyl propionate, ethyl propionate, butyl propionate and isopentyl propionate, and preferably ethyl propionate.

An acetate ester is specifically exemplified by methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate and benzyl acetate, preferably methyl acetate, ethyl acetate, propyl acetate, isopropyl acetate, butyl acetate, isobutyl acetate, sec-butyl acetate, pentyl acetate, isopentyl acetate, sec-hexyl acetate and cyclohexyl acetate, more preferably methyl acetate, ethyl acetate, propyl acetate, isopropyl acetate, butyl acetate and isobutyl acetate, and most preferably ethyl acetate.

A formate ester is specifically exemplified by methyl formate, ethyl formate, propyl formate, isopropyl formate, butyl formate, isobutyl formate, sec-butyl formate and pentyl formate, preferably methyl formate, ethyl formate, propyl formate, butyl formate, isobutyl formate and pentyl formate, and most preferably ethyl formate.

An ether solvent may be cyclic or non-cyclic and saturated or unsaturated, is not particularly restricted, and a saturated ether solvent is preferably used. In general, a C₃₋₂₀ ether solvent is used, a C₄₋₁₂ ether solvent is preferably used, and a C₄₋₈ ether solvent is more preferably used. An ether solvent is specifically exemplified by diethyl ether, methyl tert-butyl ether, dipropyl ether, diisopropyl ether, dibutyl ether, dihexyl ether, ethyl vinyl ether, butyl vinyl ether, anisole, phenetol, butyl phenyl ether, methoxytoluene, dioxane, furan, 2-methylfuran, tetrahydrofuran, tetrahydropyran, ethylene glycol dimethyl ether, ethylene glycol diethyl ether, ethylene glycol dibutyl ether, ethylene glycol monomethyl ether, ethylene glycol monoethyl ether and ethylene glycol monobutyl ether, preferably diethyl ether, methyl tert-butyl ether, dip ropyl ether, diisopropyl ether, dibutyl ether, dihexyl ether, anisole, phenetol, butyl phenyl ether, methoxytoluene, dioxane, 2-methylfuran, tetrahydrofuran, tetrahydropyran, ethylene glycol dimethyl ether, ethylene glycol diethyl ether, ethylene glycol dibutyl ether, ethylene glycol monomethyl ether and ethylene glycol monoethyl ether, more preferably diethyl ether, methyl tert-butyl ether, anisole, dioxane, tetrahydrofuran, ethylene glycol monomethyl ether and ethylene glycol monoethyl ether, more preferably diethyl ether, methyl tert-butyl ether and anisole, and most preferably methyl tert-butyl ether.

An alcohol solvent may be cyclic or non-cyclic and saturated or unsaturated, is not particularly restricted, and a saturated alcohol solvent is generally used. In general, a C₁₋₂₀ alcohol solvent is used, a C₁₋₁₂ alcohol solvent is preferably used, and a C₁₋₆ alcohol solvent is more preferably used. In particular, a C₁₋₅ monovalent alcohol solvent, a C₂₋₅ divalent alcohol solvent and a C₃ trivalent alcohol solvent are preferred.

An alcohol solvent is specifically exemplified by a monovalent alcohol such as methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol and 4-methylcyclohexanol; a divalent alcohol solvent such as 1,2-ethanediol, 1,2-propanediol, 1,3-propanediol, 1,2-butanediol, 1,3-butanediol, 1,4-butanediol, 2,3-butanediol and 1,5-pentanediol; and a trivalent alcohol such as glycerin.

A monovalent alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, benzyl alcohol, cyclohexanol, 1-methylcyclohexanol, 2-methylcyclohexanol, 3-methylcyclohexanol or 4-methylcyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol or cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol or neopentyl alcohol, particularly preferably methanol, ethanol, 1-propanol, 2-propanol, 1-butanol, 2-butanol, isobutyl alcohol, 2-methyl-1-butanol or isopentyl alcohol, and most preferably 2-propanol.

A divalent alcohol is preferably 1,2-ethanediol, 1,2-propanediol or 1,3-propanediol, and most preferably 1,2-ethanediol. A trivalent alcohol is preferably glycerin.

A fatty acid solvent is exemplified by formic acid, acetic acid and propionic acid, preferably formic acid and acetic acid, and most preferably acetic acid.

A ketone solvent is not particularly restricted, and a C₃₋₆ ketone solvent is preferably used. A ketone solvent is specifically exemplified by acetone, methyl ethyl ketone, methyl butyl ketone and methyl isobutyl ketone, preferably acetone and methyl ethyl ketone, and most preferably acetone.

A nitrile solvent may be cyclic or non-cyclic and saturated or unsaturated, is not particularly restricted, and a saturated nitrile solvent is generally used. In general, a C₂₋₂₀ nitrile solvent is used, a C₂₋₁₂ nitrile solvent is preferably used, and a C₂₋₈ nitrile solvent is more preferably used.

A nitrile solvent is specifically exemplified by acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisonitrile, phthalonitrile, bromotolunitrile, methyl cyanobenzoate, methoxybenzonitrile, acetylbenzonitrile, naphthonitrile, biphenylcarbonitrile, phenylpropionitrile, phenylbutyronitrile, methylphenylacetonitrile, diphenylacetonitrile, naphthylacetonitrile, nitrophenylacetonitrile, chlorobenzylcyanide, cyclopropanecarbonitrile, cyclohexanecarbonitrile, cycloheptanecarbonitrile, phenylcyclohexanecarbonitrile and tolylcyclohexanecarbonitrile.

Among them, a nitrile solvent is preferably acetonitrile, propionitrile, succinonitrile, butyronitrile, isobutyronitrile, valeronitrile, methyl cyanoacetate, ethyl cyanoacetate, benzonitrile, tolunitrile or chloropropionitrile, more preferably acetonitrile, propionitrile, butyronitrile or isobutyronitrile, and most preferably acetonitrile.

A nitrogen compound solvent except for a nitrile solvent is exemplified by an amide solvent such as formamide, N-methylformamide, N,N-dimethylformamide, N,N-dimethylacetamide and N-methylpyrrolidone; nitromethane; triethylamine; and pyridine.

A sulfur compound solvent is exemplified by dimethyl sulfoxide and sulfolane.

It is preferred to select an organic solvent used for the extraction of coenzyme Q10 described above in consideration of the boiling point, melting point, viscosity, or the like. For example, the boiling point is preferably included in the range of about 30 to 150° C. at 1 atmosphere, since a solvent having such a boiling point can be moderately heated for increasing a solubility and the solvent can be easily removed and exchanged. The melting point may be about 0° C. or higher, preferably about 10° C. or higher, and more preferably about 20° C. or higher, since a solvent having such a melting point is hardly solidified in the case of using the solvent at room temperature and cooling the solvent to room temperature or lower. The viscosity is preferably low as about 10 cP or lower at 20° C.

In the production method of the present invention, a hydrophobic organic solvent is used as a solvent at the time of cooling in a cooling step described later. Therefore, an organic solvent used for the above-described extraction of coenzyme Q10 from the coenzyme Q10-producing microorganism is preferably the same hydrophobic organic solvent as the solvent in the above cooling, thereby negating the need for solvent exchange and the like in the subsequent steps.

The hydrophobic organic solvent used for the extraction of coenzyme Q10 is not particularly restricted, and a hydrophobic solvent among the above-described organic solvents may be used. The hydrophobic organic solvent is preferably exemplified by a hydrocarbon solvent, a fatty acid ester solvent and an ether solvent, and more preferably a fatty acid ester solvent and a hydrocarbon solvent. These solvents may be used alone or in combination of two or more. An aliphatic hydrocarbon solvent is even more preferably used. As the aliphatic hydrocarbon solvent, a C₅₋₈ aliphatic hydrocarbon solvent is preferably used. The C₅₋₈ aliphatic hydrocarbon solvent is specifically exemplified by pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane and ethylcyclohexane, particularly preferably hexane, heptane and methylcyclohexane, and most preferably hexane. Further, as the aliphatic acid ester solvent, ethyl acetate is preferably used.

The hydrophobic organic solvent used for the extraction of coenzyme Q10 may contain the above-described hydrophobic solvent as a main component, and for example, may contain a small amount of a hydrophilic organic solvent or a surfactant. Such a hydrophilic organic solvent is exemplified by an alcohol solvent such as isopropanol. This further enhances the extraction efficiency of coenzyme Q10. The “contain the above-described hydrophobic solvent as a main component” as referred to herein means that the ratio of the volume of the above-described hydrophobic organic solvent to the entire solvent volume is 50 vol % or more (preferably 60 vol % or more).

In the production method of the present invention, an amount of the extraction solvent is not particularly restricted, and the concentration to the entire solution volume at the time of the extraction is preferably in the range of 25 to 80 vol %, and more preferably in the range of 50 to 75 vol %.

In addition, in the production method of the present invention, a temperature at the time of the extraction is not particularly restricted and may be generally in the range of 0 to 60° C. and preferably in the range of 20 to 50° C.

An extraction method may be any one of batch extraction and continuous extraction, and continuous extraction is industrially preferred in terms of a productivity. Among continuous extraction, countercurrent multistep extraction is particularly preferred. A stirring time in the case of batch extraction is not particularly restricted and may be generally 5 minutes or longer. An average residence time in the case of continuous extraction is not particularly restricted and may be generally 10 minutes or longer.

The concentration of coenzyme Q10 in the above-described extract is preferably 0.1 g/L or more, more preferably 1 g/L or more, further preferably 10 g/L, and even further preferably 20 g/L from the viewpoint of precipitating impurities as a solid in a necessary amount. The upper limit thereof is also not particularly restricted, and is preferably about 300 g/L, more preferably 150 g/L or less, and further preferably 100 g/L or less from the viewpoint of reducing a loss of coenzyme Q10. However, when a concentration described below is conducted, the concentration of coenzyme Q10 in the extract is not restricted to the above range, and is preferably 0.01 g/L or more, more preferably 0.1 g/L or more, further preferably 0.5 g/L, and even further preferably 1 g/L from the viewpoint of extraction efficiency of coenzyme Q10. The upper limit thereof is also not particularly restricted, and is preferably about 30 g/L, more preferably 15 g/L or less, and further preferably 10 g/L or less.

### (3) Concentrate of the Above-Described Extract as Needed

In the production method of the present invention, a concentrated liquid obtained by concentrating the above-described extract of the coenzyme Q10-producing microorganism as needed may be used. For example, as one preferred way, a large amount of the organic solvent may be used at the time of the extraction to increase the stability of the extraction operation and the extraction rate, and the extract may be appropriately concentrated before a cooling step to adjust the concentration to be suitable for solid precipitation. Examples of the concentration method in this case, but are not particularly restricted to, include evaporation concentration, membrane concentration, freeze concentration, vacuum concentration, separation through ultrasonic atomization, and a combination thereof. The degree of concentration is also not particularly restricted, but from the viewpoint of precipitating impurities as a solid in a necessary amount, the concentration of coenzyme Q10 in the concentrated liquid is, as in the case of the above-described extract, preferably 0.1 g/L or more, more preferably 1 g/L or more, further preferably 10 g/L, and even further preferably 20 g/L. The upper limit thereof is also not particularly restricted, and is preferably about 300 g/L, more preferably 150 g/L or less, and further preferably 100 g/L or less from the viewpoint of reducing a loss of coenzyme Q10. When the obtained hydrophobic organic solvent extract of the coenzyme Q10-producing microorganism satisfies the above preferred range, a concentration is not necessarily needed.

### (4) Alkaline Treatment and Concentration of the Above-Described Extract as Needed

Furthermore, in the production method of the present invention, it is preferred to conduct an alkaline treatment in combination with a concentration before subjecting the extract obtained by the above (2) to a cooling step. That is, as a preferred embodiment of the present invention, an extract obtained by extraction from a coenzyme Q10-producing microorganism in a hydrophobic organic solvent is brought into contact and mixed with an aqueous alkaline solution and then washed with water, the resulting extract is concentrated to obtain the concentrated liquid, and the concentrated liquid is subjected to a cooling step. By subjecting the above extract to the alkaline treatment, a fat-soluble component such as a fatty acid, fatty acid ester, or phospholipid derived from a microorganism is saponified and transferred to the aqueous phase, so that an impurity removal efficiency can be further improved and a load on a subsequent step can be reduced.

Examples of the aqueous alkaline solution with which the extract of the coenzyme Q10-producing microorganism is brought into contact and mixed include aqueous ammonia, an aqueous sodium hydroxide solution, an aqueous potassium hydroxide solution, an aqueous lithium hydroxide solution, an aqueous sodium carbonate solution, an aqueous sodium hydrogen carbonate solution, a magnesium oxide aqueous solution, an aqueous calcium hydroxide solution, an aqueous sodium acetate solution, and the like. A strong alkali is preferable in view of saponification efficiency, and an aqueous sodium hydroxide solution and an aqueous potassium hydroxide solution are more preferable also in consideration of economic efficiency. The concentration of the aqueous alkaline solution used depends on the type of alkali used and cannot be defined categorically. For example, when a strong alkali is used, the concentration of the aqueous alkaline solution is preferably 0.1 to 20 wt %, and more preferably 2 to 10 wt %. The amount of the aqueous alkaline solution to be brought into contact with the extract is not particularly restricted, and is, for example, 1 to 200 vol %, preferably 1 to 30 vol %, and more preferably 1 to 10 vol %, relative to the extract.

The contact of the extract with the aqueous alkaline solution may be conducted in any one of a batch manner and a continuous manner. Industrially, the contact in a continuous manner is preferable in terms of productivity, and the contact in a cocurrent type continuous manner is particularly preferable in view of cleaning. The stirring time in the contact in a batch manner is not particularly restricted, and is usually 1 minute or longer. The average residence time in the contact in a continuous manner is not particularly restricted, and is usually 10 seconds or longer.

When the extract after the contact with the aqueous alkaline solution is directly used, the extract is likely to suffer deterioration in the quality due to heat or the like, such as decomposition of coenzyme Q10 and formation of a dimer, and therefore is preferably washed with water. The amount of water to be brought into contact with the extract is not particularly restricted, and is 1 to 200 vol %, preferably 1 to 30 vol %, and more preferably 1 to 10 vol %, relative to the extract.

The contact of the extract with water may be conducted in any one of a batch manner and a continuous manner. Industrially, the contact in a continuous manner is preferable in terms of productivity, and the contact in a cocurrent type continuous manner is particularly preferable in view of cleaning. The stirring time in the contact in a batch manner is not particularly restricted, and is usually 1 minute or longer. The average residence time in the contact in a continuous manner is not particularly restricted, and is usually 10 seconds or longer.

Preferably, the extract after washing with water is appropriately concentrated, and the concentrated liquid is subjected to a subsequent cooling step. The concentration method of the extract and its preferable concentration are as described above.

### (5) Control of Water Content of Hydrophobic Organic Solvent Extract of Coenzyme Q10-Producing Microorganism or Concentrated Liquid Of The Hydrophobic Organic Solvent Extract

When the extract or the concentrated liquid of the coenzyme Q10-producing microorganism obtained as described above is one obtained particularly by extraction from a culture solution of a microorganism, wet microorganism cells, an aqueous dispersion of microorganism cells, or a homogenate thereof, the extract contains water derived from those. In the production method of the present invention, when a hydrophobic organic solvent is used as an extraction solvent, or a small amount of a hydrophilic organic solvent is used in combination with a hydrophobic organic solvent as an extraction solvent, the hydrophobic organic solvent extract or the concentrated liquid of the hydrophobic organic solvent extract having a water content of 50 ppm by weight or more and 1% by weight or less can be directly subjected to the subsequent cooling step. If the water content does not satisfy the above range, water may be added to or removed from the hydrophobic organic solvent extract or the concentrated liquid of the hydrophobic organic solvent extract as appropriate before the cooling step. On the other hand, when a hydrophilic organic solvent is used as an extraction solvent, the hydrophilic organic solvent may be exchanged with a hydrophobic organic solvent before the cooling step, then the water content may be adjusted as needed, and the cooling step may be performed.

Note that, in this disclosure, any extract of a coenzyme Q10-producing microorganism in which the solvent is exchanged with another hydrophobic organic solvent is also referred to as a “hydrophobic organic solvent extract of a coenzyme Q10-producing microorganism” for convenience. Furthermore, in the present invention, some amount of impurities may be removed from the above-described hydrophobic organic solvent extract of the coenzyme Q10-producing microorganism by another method, and the water content thereof may be adjusted as needed.

### (6) Cooling Step

In the production method of the present invention, the hydrophobic organic solvent extract of the coenzyme Q10-producing microorganism or the concentrated liquid of the hydrophobic organic solvent extract obtained as described above is cooled to precipitate impurities derived from microorganisms other than coenzyme Q10 as a solid. In this cooling step, the extract or the concentrated liquid to be cooled contains a hydrophobic organic solvent as a solvent as described above, and the water content of the extract or the concentrated liquid needs to be controlled in the range of 50 ppm or more and 1% or less in terms of % by weight. If the water content is less than 50 ppm, a removal rate of components other than coenzyme Q10 in the cooling step may decrease. On the other hand, if the water content is more than 1%, a large amount of water is contained in the solution even after the cooling step, which may adversely affect the subsequent steps such as column chromatography. The upper limit of the water content is preferably 0.4% or less, more preferably 0.3% or less, and further preferably 0.2% or less.

As described repeatedly, the solvent of the extract or the concentrated liquid used at the time of the cooling is not particularly restricted as long as it is a hydrophobic organic solvent. When a hydrophobic organic solvent is used as an extract of a coenzyme Q10-producing microorganism, the hydrophobic organic solvent can be used in the cooling step as it is. Even when a small amount of a hydrophilic organic solvent is used in combination at the time of the extraction, the hydrophilic organic solvent may be present in the extract or the concentrated liquid in the cooling step as long as it does not interfere with the precipitation of solid (for example, within the range of 5 vol % or less). When the alkali treatment is performed as the above-described preferred embodiment, the hydrophilic organic solvent used in combination at the extraction is transferred to the aqueous phase. Therefore, in many cases, the hydrophilic organic solvent is removed to the extent that it does not hinder the cooling step. Furthermore, another hydrophobic organic solvent may be added to and mixed with the extract of the coenzyme Q10-producing microorganism, or the extract in which the solvent has been exchanged with another hydrophobic organic solvent may be used. The hydrophobic organic solvent at the time of the cooling is specifically exemplified by a hydrocarbon solvent, a fatty acid ester solvent, an ether solvent, and a nitrogen compound solvent such as a nitrile solvent and an amide solvent. Among these, a hydrocarbon solvent, a fatty acid ester solvent, and an ether solvent are preferably used, a hydrocarbon solvent and a fatty acid ester solvent are more preferably used, an aliphatic hydrocarbon solvent can be further preferably used. As specific examples or more preferable examples thereof, those described as the extraction solvent at the time of the extraction can be exemplified.

The cooling temperature at the time of the cooling is appropriately selected as needed within the range of room temperature of, for example, 25° C. or lower, preferably 20° C. or lower, more preferably 15° C. or lower, further preferably 5° C. or lower, and still more preferably 2° C. or lower. The lower limit of the cooling temperature is, for example, −5° C. from the viewpoint of preventing a loss of coenzyme Q10 and considering that a large amount of energy is required for cooling. The cooling rate is not particularly restricted, and is preferably 100° C./h or lower, more preferably 50° C./h or lower, and further preferably 30° C./h or lower.

In the production method of the present invention, the purity of coenzyme Q10 in a filtrate can be improved by precipitating a larger amount of solid in the cooling. The concentration of solid to be precipitated is not particularly restricted, and is, for example, 1 g/L or more, preferably 1.5 g/L or more, and more preferably 2 g/L or more.

As described above, the above-described extract, the extract that has been washed with water after the contact and mixing with the aqueous alkaline solution, and the concentrated liquid thereof contain a slight amount of water. This water must be prevented from flowing into subsequent steps such as column chromatography and crystallization as much as possible. In the present invention, when the extract or the concentrated liquid obtained as described above is cooled to precipitate a solid, water can be removed together with the solid. Depending on the water concentration in the extract before the separation and removal of the solid, the filtrate after the extract has been cooled, and a sufficient amount of the solid has been precipitated has a water concentration of usually 300 ppm by weight or less, preferably 200 ppm by weight or less, and more preferably 100 ppm by weight or less.

### (7) Separation Step of Solid

After the cooling as described above, the precipitated solid is separated and removed. In the production method of the present invention, the method for separating and removing the precipitated solid is not particularly restricted as long as it can separate and remove a solid. For example, a common filtration method using a paper filter, cloth filter, cylindrical filter or the like, and in addition, separation methods such as natural sedimentation separation, centrifugation, membrane separation, vibratory membrane separation, separation using a liquid cyclone or a rotary filter, and adsorption separation, or a combination of these separation methods can be used.

In order to industrially produce coenzyme Q10 efficiently by taking into consideration a separation and removal performance of solid and a required power thereof, downsizing of a separation and removal apparatus, and operation load, it is preferred to use a rotary filter. The material of the rotary filter is not particularly restricted, and examples thereof include synthetic resins such as polyethylene, polypropylene, polymethylmethacrylate, polystyrene, and fluororesin, and composites thereof; oxide ceramics such as alumina, zirconia, barium titanate, titanium oxide, and composites thereof, hydroxide ceramics such as hydroxyapatite, carbide ceramics such as silicon carbide, nitride ceramics such as silicon nitride, halide ceramics such as fluorite, and phosphate ceramics; and metals such as iron, copper, zinc, tin, mercury, lead, aluminum, stainless steel, and composites thereof. The pore size of the rotary filter is not particularly restricted, and is preferably 1 nm to 2 μm in order to separate a desired solid, and is preferably 60 nm to 1 μm in consideration of throughput and ease of cleaning. Specific examples of the rotary filter include “Ceramic Rotary Filter” manufactured by Hiroshima Metal & Machinery Co., Ltd., “Ceramic Rotary Filter” manufactured by Eurotech company, and “Mitsubishi Dyna Filter” manufactured by Mitsubishi Kakoki Co., Ltd.

In the production method of the present invention, the separation and removal of solid can be conducted in any of a batch manner, a semi-batch manner, and a continuous manner. The filtration can be conducted also by any method of a circulation filtration and a dead-end filtration. For example, the separation and removal of solid can be conducted by the following method: a given amount of the extract or the concentrated liquid thereof is reserved before or after the cooling, then the precipitated solid is separated and removed, a filtrate is obtained at an arbitrary ratio, and at the same time, the separated and removed solid is returned to the previous step; or the extract or the concentrated liquid thereof is continuously cooled and subjected to separation and removal of solid, and the operations of obtaining a filtrate and returning the separated and removed solid to the previous step are continuously conducted. In either method, the treatment rate at the separation and removal of the solid is not particularly restricted, and is usually 5 L/h or more, preferably 50 L/h or more, and more preferably 150 L/h or more. The obtained filtrate containing coenzyme Q10 can be used as it is also in the next step.

In order to prevent a loss of coenzyme Q10, the separated and removed solid may be returned to the step prior to the previous cooling step and treated repeatedly. Specifically, it is preferred to add the separated and removed solid to the extract before the above-described contact and mixing with the aqueous alkaline solution and perform the subsequent steps similarly. Alternatively, it is preferred to add the separated and removed solid to the step after the above-described contact and mixing with the aqueous alkaline solution (specifically, the step being either of the extract after the contact and mixing, the extract after the contact and mixing and before washing with water, or the extract after washing with water and before the concentration) and perform the subsequent steps similarly. More preferably, it is recommended to repeat a procedure in which the separated and removed solid is added to the extract before the contact and mixing with the aqueous alkaline solution, or the separated and removed solid is added to the extract after the contact and mixing with the aqueous alkaline solution and before washing with water, and the subsequent steps are conducted.

When returning to the previous step, without performing complete solid-liquid separation, the precipitated solid in a slurry state may be returned to the previous step together with a part of the extract or the concentrated liquid.

According to the above-described production method of the present invention, it is possible to minimize a loss of coenzyme Q10, and for example, the final yield of coenzyme Q10 of 99% or more can be preferably achieved. The amount of the solid or the slurry containing the solid to be returned to the previous step is not particularly restricted, and is preferably 0.1 to 50 vol %, more preferably 0.1 to 10 vol %, and further preferably 0.1 to 5 vol %, relative to the volume of the solution before the separation and removal of the solid.

Furthermore, for example, when a rotary filter is used as a method for separating and removing the solid, the loss of coenzyme Q10 can be further reduced by regularly washing the rotary filter with the same solvent as the solvent of the extract or the concentrated liquid and returning also the washing solution to the previous step.

In the production method of the present invention, impurities to be separated and removed as the solid include fat-soluble components other than coenzyme Q10 derived from the coenzyme Q10-producing microorganism. As such fat-soluble components, a sterol derivative and a fat and oil component can be mainly given.

The sterol derivative is not particularly restricted and exemplified by cholesterol, camp esterol, desmosterol, brassicasterol, stigmasterol, α-sitosterol, β-sitosterol, dihydro-β-sitosterol, γ-sitosterol, 7-dehydrocholesterol, ergosterol and dihydroergosterol. In addition, a sterol ester having an ester bond at the end of each of these sterol derivatives is also included in the above-described sterol derivative. Among these, a plurality of sterol derivatives can be also separated and removed in the production method of the present invention. The production method of the present invention is advantageous in that it is possible to selectively separate a sterol fatty acid ester, which is contained in a large amount in a culture product after yeast or the like is cultivated.

In the production method of the present invention, a purity improvement percent point of coenzyme Q10 in the solution after the separation and removal of solid in comparison with the solution before the separation and removal of solid is generally 2 percent points or more, and preferably 2.5 percent points or more. The upper limit thereof is not particularly restricted, and may be 10 percent points or less, or 7 percent points or less. The purity improvement percent point corresponds to the difference of weight percent of Q10 in two non-volatile constituents obtained by drying the solutions before and after the separation and removal of solid.

As for a removal rate of impurities removed as a solid, for example, a removal rate of ergosterol is usually 20 wt % or more, preferably 30 wt % or more, and more preferably 40 wt % or more. The upper limit thereof is 100 wt % or less, and may be usually 90 wt % or less, or 60 wt % or less.

In the production method of the present invention, by the above-described procedures, coenzyme Q10 that has been purified or has improved purity can be isolated and recovered. The coenzyme Q10 solution after the separation step can be directly used or may be further treated to obtain a coenzyme Q10-containing composition or a coenzyme Q10 crystal having a more preferred form or higher purity. Such a treatment step is exemplified by concentration, solvent exchange, oxidation, reduction, column chromatography, crystallization, and a combination thereof. For example, after the separation step, a solvent may be distilled away from the coenzyme Q10 extract from which a solid has been separated and removed for concentration to obtain purified coenzyme Q10. Alternatively, after coenzyme Q10 is further purified by column chromatography using silica gel or the like as needed, an organic solvent may be distilled away to obtain purified coenzyme Q10. Furthermore, the target coenzyme Q10 may be obtained as a crystal by crystallization procedure. Before the above-described column chromatography, oxidation, reduction and crystallization, a solvent may be exchanged as needed. For example, coenzyme Q10 is extracted in a hydrophobic organic solvent from the coenzyme Q10-producing microorganism, the obtained extract containing coenzyme Q10 is subjected to the cooling step, and then the precipitated solid is separated and removed for purification by the production method of the present invention. Oxidation or reduction may be conducted before or after the separation and removal as needed to obtain highly pure coenzyme Q10 as a crystal by a crystallization procedure.

In the production method of the present invention, in order to produce only reduced coenzyme Q10 or coenzyme Q10 of which reduced coenzyme Q10 ratio is high, only reduced coenzyme Q10 or coenzyme Q10 of which reduced coenzyme Q10 ratio is high can be obtained without special treatment by using a microorganism which can produce coenzyme Q10 having high reduced coenzyme Q10 ratio as the coenzyme Q10-producing microorganism and conducting the above-described extraction, cooling, and separation and removal of solid under an antioxidant atmosphere, for example, under an inert atmosphere such as nitrogen gas. It is possible that a reduced coenzyme Q10 ratio can be further increased by reducing the thus obtained coenzyme Q10 having high reduced coenzyme Q10 ratio. In addition, it is also possible that the coenzyme Q10-containing extract is not especially subjected to antioxidant means or is oxidized by oxygen in the air and an oxidant to obtain the coenzyme Q10-containing extract having relatively low reduced coenzyme Q10 ratio such as not more than 50 mol % or not more than 30 mol %, and the obtained coenzyme Q10-containing extract having relatively low reduced coenzyme Q10 ratio is subjected to the cooling, and the separation and removal of solid of the production method of the present invention, and then to a reduction reaction, thereby producing coenzyme Q10 having a high reduced coenzyme Q10 ratio. For the purpose of producing reduced coenzyme Q10, it is preferred that a reduced coenzyme Q10 ratio in the final production step or of the final product be high. The reduced coenzyme Q10 ratio in a total amount 100 mol % of coenzyme Q10 may be, for example, 70 mol % or more, preferably 80 mol % or more, more preferably 90 mol % or more, and even more preferably 96 mol % or more.

As the more specific one embodiment, coenzyme Q10 is extracted in an organic solvent from the coenzyme Q10-producing microorganism, and the thus obtained extract is washed with water after being brought into contact with an aqueous alkaline solution and then subjected to a concentration treatment to obtain a solution containing a trace amount of water. The thus obtained solution is subjected to the cooling step and the separation step of solid of the production method of the present invention, coenzyme Q10 is further purified by column chromatography and subjected to a reduction treatment as needed, and a crystal of highly pure reduced coenzyme Q10 can be obtained by crystallization.

The production method of the present invention can be also used for producing oxidized coenzyme Q10. In such a case, the coenzyme Q10 having high oxidized coenzyme Q10 ratio can be obtained by simple procedure. For example, coenzyme Q10 is extracted in an organic solvent from the microorganism cells, microorganism cell homogenate, aqueous dispersion of the microorganism cell homogenate, dried microorganism cells, or dried microorganism cell homogenate containing coenzyme Q10, and thus obtained extract may be subjected to an oxidation treatment by an oxidant before or after the cooling step and the separation step. Alternatively, the coenzyme Q10 having high oxidized coenzyme Q10 ratio can be obtained due to natural oxidation by merely conducting extraction, adsorption, other purification and aftertreatment in the air or drying the microorganism in the air before the extraction.

As the more specific one embodiment, coenzyme Q10 is extracted in an organic solvent from the coenzyme Q10-producing microorganism, the thus obtained extract containing coenzyme Q10 is subjected to an oxidation treatment as needed after or upon the contact with an aqueous alkaline solution, and subjected to the cooling step and the separation step of solid of the production method of the present invention. After the exchange of the solvent, coenzyme Q10 is further purified by column chromatography, and a crystal of highly pure oxidized coenzyme Q10 may be obtained by crystallization.

The present application claims the benefit of the priority date of Japanese patent application No. 2018-062841 filed on Mar. 28, 2018. All of the contents of the Japanese patent application No. 2018-062841 filed on Mar. 28, 2018, are incorporated by reference in its entirety herein.

### EXAMPLES

Hereinafter, the present invention is described in more detail with Examples and Comparative Examples but is not restricted to the following Examples. In addition, the yield and purity of coenzyme Q10 in Examples and Comparative Examples do not represent the limiting value of the present invention nor the upper limit. The concentration of coenzyme Q10 was measured by high-performance liquid chromatography (HPLC) (manufactured by SHIMADZU) in the following condition.

### HPLC Measurement Condition

### Column: YMC-Pack ODS-A

Oven temperature: 30° C.
Mobile phase: methanol/hexane=85/15 by volume
Flow rate: 1.0 ml/min

### Detection: UV 275 nm

A purity improvement percent point (% pt.) of coenzyme Q10 was calculated as the difference of weight percent of Q10 in two non-volatile constituents obtained by drying the solutions before and after the separation and removal of solid.

A removal rate of ergosterol was calculated by measuring ergosterol concentrations in the solutions before and after the separation and removal of solid and using the following formula. The ergosterol concentration is described as ERG concentration and was measured by using HPLC in the same condition as the above-described measurement condition of coenzyme Q10 concentration.

Removal rate of ergosterol (ERG)={(ERG concentration before the separation and removal of solid−ERG concentration after the separation and removal of solid)/(ERG concentration before the adsorption treatment)}×100.

The water content was measured using Karl Fischer (AQUACOUNTER AQ-2100 manufactured by Hiranuma Sanygo Co., Ltd.).

### Example 1

Saitoella complicata IFO10748 strain, which could produce coenzyme Q10, was aerobically cultivated in a culture medium (peptone 5 g/L, yeast extract 3 g/L, malt extract 3 g/L, glucose 20 g/L, pH 6.0) at 25° C. for 160 hours. The obtained microorganism culture medium containing coenzyme Q10 was concentrated by centrifugation, and the microorganism was homogenized by a pressure homogenizer. To the obtained microorganism homogenate were added 1.8 times amount of hexane and 0.7 times amount of 2-propanol to the volume of the microorganism homogenate, and the mixture was stirred at 40° C. for 1 hour to extract coenzyme Q10. To the extract of the coenzyme Q10-producing microorganism were added 8 vol % of a 4 wt % aqueous solution of sodium hydroxide relative to the extract, and 0.5 vol % of a 7% aqueous hydrogen peroxide relative to the extract as an oxidant. The mixture was stirred for 3 minutes and then allowed to stand to separate an aqueous layer. To the extract after the separation was added 13 vol % of tap water relative to the extract, and the resulting extract was stirred and washed with water. After repeating the washing twice, the extract was concentrated so that the coenzyme Q10 concentration was adjusted to 50 g/L. The reduced coenzyme Q10 ratio in the concentrate, i.e. the ratio of reduced coenzyme Q10 in the total coenzyme Q10, was 0 wt %. The concentrated liquid having a water content of 663.4 ppm by weight (the same applies hereinbelow) was cooled to 20° C. and subjected to suction filtration to separate and remove a solid. For the filtration, a Kiriyama funnel and No. 5-C filter paper for use in the Kiriyama funnel were used. As a result of analysis of the filtrate, it was confirmed that the water content in the filtrate was 201.8 ppm, the removal rate of ergosterol was 34.8%, and the purity of coenzyme Q10 was increased by 4.0% pt.

### Example 2

The same concentrated liquid (water content: 663.4 ppm) of the coenzyme Q10-producing microorganism as that of Example 1 was cooled to 15° C. and subjected to suction filtration in the same manner as in Example 1 to separate and remove a solid. As a result of analysis of the filtrate, it was confirmed that the water content was 165.44 ppm, the removal rate of ergosterol was 39.0%, and the purity of coenzyme Q10 was increased by 4.9% pt.

### Example 3

The same concentrated liquid (water content: 663.4 ppm) of the coenzyme Q10-producing microorganism as that of Example 1 was cooled to 10° C. and subjected to suction filtration in the same manner as in Example 1 to separate and remove a solid. As a result of analysis of the filtrate, it was confirmed that the water content was 107.2 ppm, the removal rate of ergosterol was 46.3%, and the purity of coenzyme Q10 was increased by 5.3% pt.

### Example 4

The same concentrated liquid (water content: 663.4 ppm) of the coenzyme Q10-producing microorganism as that of Example 1 was cooled to 2° C. and subjected to suction filtration in the same manner as in Example 1 to separate and remove a solid. As a result of analysis of the filtrate, it was confirmed that the water content was 63.8 ppm, the removal rate of ergosterol was 51.0%, and the purity of coenzyme Q10 was increased by 5.8% pt.

### Example 5

The concentrated liquid (water content: 585 ppm) of the coenzyme Q10-producing microorganism was prepared in the same manner as in Example 1, cooled to 10° C., and subjected to centrifugation at 9000 g for 5 minutes to recover a supernatant. Allegra X-22R CENTRIGUGE manufactured by Beckman Coulter, Inc. was used for the centrifugation. It was confirmed that the water content in the concentrated liquid was 585 ppm, whereas the water content in the recovered supernatant was reduced to 109.5 pp.

### Example 6

The concentrated liquid (water content: 121.8 ppm) of the coenzyme Q10-producing microorganism was prepared in the same manner as in Example 1, cooled to 17° C., and subjected to separation so that the volume ratio of a slurry containing a solid and a filtrate became 1:9 by a separation method using Mitsubishi Dyna Filter (manufactured by Mitsubishi Kakoki Co., Ltd.) having one aluminum oxide disc with an average pore size of 0.2 gm and a filtration area of 0.034 square meters. As a result of analyses of the concentrated liquid and the filtrate, it was confirmed that the water content in the concentrated liquid was 121.8 ppm, whereas the water content in the filtrate was reduced to 67.1 ppm, the removal rate of ergosterol was 68.1%, and the purity of coenzyme Q10 was increased by 4.7% pt.

### Example 7

The same concentrated liquid (water content: 121.8 ppm) of the coenzyme Q10-producing microorganism as that of Example 6 was cooled to 15° C., and subjected to separation so that the volume ratio of a slurry containing a solid and a filtrate became 1:9 by a separation method using Mitsubishi Dyna Filter (manufactured by Mitsubishi Kakoki Co., Ltd.). As a result of analyses of the filtrate, the water content was reduced to 54.7 ppm, the removal rate of ergosterol was 69.8%, and the purity of coenzyme Q10 was increased by 4.5% pt.

### Example 8

The balance of coenzyme Q10 was measured from the coenzyme Q10 concentrations in the concentrated liquid of the coenzyme Q10-producing microorganism used in Example 7, the obtained filtrate, the slurry containing a solid after solid-liquid separation, and the washing solution of the apparatus. As a result, it was confirmed that 100% recovery of coenzyme Q10 was possible without a loss of coenzyme Q10 by the separation.

### Example 9

The concentrated liquid (509.9 ppm) of the coenzyme Q10-producing microorganism was prepared in the same manner as in Example 1, cooled to 15° C., and subjected to separation so that the volume ratio of a slurry containing a solid and a filtrate became 1:2.6 by a separation method using Ceramic Rotary Filter (manufactured by Hiroshima Metal & Machinery Co., Ltd.,) having one aluminum oxide disc with an average pore size of 0.2 gm and a filtration area of 0.0334 square meters. As a result of analyses of the concentrated liquid and the filtrate, it was confirmed that the water content in the concentrated liquid was 509.9 ppm, whereas the water content in the filtrate was reduced to 133.2 ppm, the removal rate of ergosterol was 69.1%, and the purity of coenzyme Q10 was increased by 7.0% pt.

### Example 10

To the extract obtained in the same manner as in Example 1 was added 0.5 vol % of the slurry containing a large amount of solid after the separation obtained in Example 7 relative to the extract, the mixture was brought into contact and mixed with an aqueous alkaline solution in the same manner as in Example 1 and then washed with water, and the aqueous layer and the extract were separated. The coenzyme Q10 concentration in the aqueous layer was 0.01 wt % or less, and thus there was no loss of coenzyme Q10. There were also no problems with the steps of alkali treatment and washing. From the above, it was confirmed that there is no problem in returning the slurry containing a large amount of solid to the step before being brought into contact and mixed with the aqueous alkaline solution.

### Example 11

To the extract obtained in the same manner as in Example 1 was added 2 vol % of the slurry containing a large amount of solid after the separation obtained in Example 7 relative to the extract, the mixture was brought into contact and mixed with an aqueous alkaline solution in the same manner as in Example 1 and then washed with water, and the aqueous layer and the extract were separated. The coenzyme Q10 concentration in the aqueous layer was 0.01 wt % or less, and thus there was no loss of coenzyme Q10. There were also no problems with the steps of alkali treatment and washing. From the above, it was confirmed that there is no problem in returning the slurry containing a large amount of solid to the step before being brought into contact and mixed with the aqueous alkaline solution.

### Example 12

Into the extract obtained by the same method as in Example 1 were poured 0.5 vol % of the slurry containing a large amount of solid after the separation obtained in Example 7 and 8 vol % of a 4 wt % aqueous solution of sodium hydroxide relative to the extract. The extract and the aqueous layer were separated in a settler, a continuous operation of continuously discharging the aqueous layer was conducted for 2 hours. Throughout the operation, ease of separation between the extract and the aqueous layer in the settler was satisfactory. The coenzyme Q10 concentration in the aqueous layer recovered after the completion of the operation was 0.002 wt %, and thus there was no loss of coenzyme Q10. There were also no problems with the above-described steps. From the above, it was confirmed that there is no problem in returning the concentrate containing a large amount of solid to the step before being brought into contact and mixed with the aqueous alkaline solution.

### Comparative Example 1

The same concentrated liquid (water content: 663.4 ppm) of the coenzyme Q10-producing microorganism as that of Example 1 was placed in an environment of 40° C. for 2 hours and subjected to suction filtration in the same manner as in Example 1 to separate and remove a solid. As a result of analysis of the filtrate, it was confirmed that although the purity of coenzyme Q10 was increased by 3.2%pt., the water content in the filtrate was 334.7 ppm, and the removal rate of ergosterol was 2.9%.

### Comparative Example 2

The same concentrated liquid (water content: 585 ppm) of the coenzyme Q10-producing microorganism as that of Example 5 was, under the condition of 30° C. without cooling, subjected to centrifugation at 9000 g for 5 minutes using the same centrifuge as that in Example 5 to recover a supernatant. It was confirmed that the water content in the recovered supernatant was reduced to 309.5 ppm.

### Comparative Example 3

The concentrated liquid (water content: 550.5 ppm) of the coenzyme Q10-producing microorganism was prepared in the same manner as in Example 1. To the prepared extract were added 3.5 wt % of activated clay and 4 wt % of a filter aid (Rokahelp: manufactured by Mitsui Mining & Smelting Co., Ltd.), and the mixture was stirred at 40° C. and subjected to suction filtration to separate and remove a solid. For the filtration, a Kiriyama funnel and No. 5-C filter paper for use in the Kiriyama funnel were used. As a result, it was confirmed that the water content in the concentrated liquid was 550.5 ppm, whereas the water content in the filtrate was 402.5 ppm, the yield of coenzyme Q10 was 97.8%, the removal rate of ergosterol was 8.8%, and the purity improvement percent point of coenzyme Q10 was 0.5% pt.

### Comparative Example 4

To the same concentrated liquid (water content: 550.5 ppm) of the coenzyme Q10-producing microorganism as that of Comparative Example 3 were added 3.5 wt % of activated clay and 4 wt % of a filter aid (Rokahelp: manufactured by Mitsui Mining & Smelting Co., Ltd.), and the mixture was stirred at 40° C. and then cooled to 18° C. Then, a solid was separated and removed in the same manner as in Comparative Example 3. As a result, it was confirmed that the water content in the filtrate was 220.5 ppm, the removal rate of ergosterol was 62.4%, and the purity of coenzyme Q10 was increased by 3.5% pt, but the yield of coenzyme Q10 was 98.4%.

1-7. (canceled)

8. A method for producing coenzyme Q10 comprising:
(a) providing a first extract, wherein the first extract comprises (i) a hydrophobic organic solvent extract having a coenzyme Q10-producing microorganism or (ii) a concentrated liquid of an initial hydrophobic organic solvent extract, and wherein the hydrophobic organic solvent extract or the concentrated liquid has a water content of 50 ppm by weight or more and 1% by weight or less;
(b) cooling the first extract under conditions effective to precipitate a solid; and
(c) separating and removing the solid that was precipitated in (b).

9. The method according to claim 8, wherein the concentrated liquid of an initial hydrophobic organic solvent extract in (a) is obtained by:
(a′) mixing the initial hydrophobic organic solvent extract having the coenzyme Q10-producing microorganism with an aqueous alkaline solution, and then washing with water to provide a washed extract,
(a″) concentrating the washed extract, to obtain the concentrated liquid of the hydrophobic organic solvent extract.

10. The method according to claim 9 comprising:
repeating a procedure in which, the solid that was separated and removed in (c), is added to a second extract having the coenzyme Q10-producing microorganisms, mixed with a second aqueous alkaline solution, washed with water, and then subject to a second cooling step; or
repeating a procedure in which, a second extract having the coenzyme Q10-producing microorganisms is mixed with a second aqueous alkaline solution, the solid that was separated and removed in (c) is added to a second extract, a second extract is washed with water, and a second extract is subject to a second cooling step.

11. The method according to claim 8, wherein the cooling is conducted at a temperature of 20° C. or lower.

12. The method according to claim 8, wherein a concentration of coenzyme Q10 in the hydrophobic organic solvent extract or the concentrated liquid at the time of the cooling in the cooling step is 0.1 g/L or more and 300 g/L or less.

13. The method according to claim 8, wherein the hydrophobic organic solvent comprises a hydrocarbon solvent, a fatty acid ester solvent, or mixtures thereof.

14. The method according to claim 8, wherein the solid is separated and removed from the first extract by a rotary filter.
